(19) United States
(12) Patent Application Publication
(10) Pub. No.: US 2022/0296687 A1
(43) Pub. Date: Sep. 22, 2022

(54) NEUROTOXIN COMPOSITIONS FOR USE IN TREATING NEUROLOGIC AND PSYCHIATRIC DISORDERS

(71) Applicant: AEON Biopharma, Inc., Irvine, CA (US)

(72) Inventor: Gregory F. Brooks, Irvine, CA (US)

(21) Appl. No.: 17/638,673

(22) PCT Filed: Aug. 28, 2020
(86) PCT No.: PCT/US2020/048628

§ 371 Date: Feb. 25, 2022

Related U.S. Application Data
(60) Provisional application No. 62/894,533, filed on Aug. 30, 2019.

(51) Int. Cl. A61K 38/48; A61P 25/22; A61P 25/24

(52) U.S. Cl. CPC A61K 38/4893; A61P 25/22; A61P 25/24

(57) ABSTRACT

Disclosed herein are compositions and methods for use in treating neurologic and psychiatric disorders.

### FIELD

The present specification relates to the use of neurotoxins administered to the Stellate Ganglion (SG) nerve collection, for example to treat neurologic and psychiatric disorders including Post-Traumatic Stress Disorder (PTSD).

### BACKGROUND

The SG is a collection of nerves found at the level of the sixth and seventh cervical vertebrae (the last vertebra of the neck). The nerves are located in front of the vertebrae. They are part of the sympathetic nervous system, supplying the head, upper extremities, and organs of the chest. Treatment, for example by “blocking” the SG (establishing an “SGB”, or a “Stellate Ganglion Block”) can be useful in treating a number of disorders, including Post-Traumatic Stress Disorder (PTSD), Depression, Anxiety, Headache, Post-Herpetic Neuralgia (PHN), Intractable Angina, Arrhythmias, and Chronic Regional Pain Syndrome (CRPS).

PTSD is a mental health condition triggered by a significant, often stressful or threatening event—either experiencing it or witnessing it. Symptoms can include flashbacks, nightmares and severe anxiety, as well as uncontrollable thoughts about the event. PTSD has been known by many names in the past, such as “shell shock” during the years of World War I and “combat fatigue” after World War II. But PTSD can occur in anyone at any age. PTSD affects approximately 3.5 percent of U.S. adults, and an estimated one in 11 people will be diagnosed with PTSD in their lifetime. Women are twice as likely as men to have PTSD.

People with PTSD can have intense, disturbing thoughts and feelings related to their experience that last long after the traumatic event has ended. They may relive the event through flashbacks or nightmares; they may feel sadness, fear or anger; and they may feel detached or estranged from other people. People with PTSD may avoid situations or people that remind them of the traumatic event, and they may have strong negative reactions to something as ordinary as a loud noise or an accidental touch.

A diagnosis of PTSD requires exposure to an upsetting traumatic event. However, exposure could be indirect rather than first hand. For example, PTSD could occur in an individual learning about the violent death of a close family. It can also occur as a result of repeated exposure to descriptions of trauma such as police officers exposed to details of child abuse cases.

Symptoms of PTSD fall into four categories. Specific symptoms can vary in severity.
a. Intrusive thoughts such as repeated, involuntary memories; distressing dreams; or flashbacks of the traumatic event. Flashbacks may be so vivid that people feel they are re-living the traumatic experience or seeing it before their eyes.
b. Avoiding reminders of the traumatic event may include avoiding people, places, activities, objects and situations that bring on distressing memories. People may try to avoid remembering or thinking about the traumatic event. They may resist talking about what happened or how they feel about it.
c. Negative thoughts and feelings may include ongoing and distorted beliefs about oneself or others (e.g., “I am bad,” “No one can be trusted”); ongoing fear, horror, anger, guilt or shame; much less interest in activities previously enjoyed; or feeling detached or estranged from others.
d. Arousal and reactive symptoms may include being irritable and having angry outbursts; behaving recklessly or in a self-destructive way; being easily startled; or having problems concentrating or sleeping.

Common PTSD co-morbidities include major depression, anxiety disorders, impulsivity/violent behavior, and substance abuse.

Sufferers of PTSD are often treated with therapy, though new modalities of treatment are gaining acceptance. For example, medications are also used in PTSD treatment, and Virtual Reality exposure can be helpful as well.

Although there are no medications that have been specifically designed to treat PTSD, there are a variety of well-established medications currently used to treat other psychiatric conditions (such as mood and anxiety disorders) that have been found to be helpful in managing PTSD symptoms. Selective serotonin reuptake inhibitors, commonly referred to as SSRIs, are a type of medication usually prescribed to help with symptoms of depression and anxiety. It is noted that SSRIs are usually the common category of medications to turn to in the treatment of PTSD. However, an SNRI can be used as well. SNRI stands for serotonin-norepinephrine reuptake inhibitor and they are often used for the treatment of depression. Not uncommonly, other categories of medications such as the atypical antipsychotics and the anti-hypertensive alpha blocker prazosin may be used to decrease PTSD symptoms.

However, all of these treatments suffer from inconsistent effectiveness as well as short duration of effect.

### SUMMARY

Disclosed herein are compositions and methods comprising neurotoxins, for example clostridial neurotoxins including botulinum toxins, and the use thereof to treat neurologic and psychiatric disorders, for example PTSD, Depression, Anxiety, Headache, PHN, Intractable Angina, Arrhythmias, and CRPS. Disclosed methods can include the use of both intra-muscular and nerve-rich administration sites, for example injection into the SG to “block” the nerve bundle, establishing an SGB.

Disclosed treatment modalities can prevent or alleviate symptoms of neurologic and psychiatric disorders. Longer duration of effect and greater reduction in symptoms as compared to anesthetic treatment alone can also be provided by the disclosed methods. Disclosed treatment methods comprise use of a neurotoxin applied to the SG or the vicinity thereof. Disclosed treatment methods comprise use of a neurotoxin in combination with or without a local anesthetic, with both applied to the SG or the vicinity thereof. Disclosed combination treatments (for example, an SSRI in combination with a neurotoxin in the case of PTSD treatment, or zoledronic acid in combination with a neurotoxin in the case of CRPS treatment) can provide a synergistic effect as compared to the effects of either administered alone.

Treatments disclosed herein can provide increased duration of relief as compared to current methods.

### BRIEF DESCRIPTION OF THE DRAWING

FIG. 1 shows the location of the Stellate Ganglion within the neck.

### DETAILED DESCRIPTION

The present disclosure is directed toward methods for reducing the occurrence and severity of symptoms associated with neurologic and psychiatric disorders including, for example, PTSD, Depression, Anxiety, Headache, PHN, Intractable Angina, Arrhythmias, and CRPS, for example through the use of a neurotoxin-induced or neurotoxin-mediated SG block, or SGB. SGB is a procedure selectively used by anesthesiologists to relieve pain. Emerging research suggests that SGB using a local anesthetic may help a subset of patients with posttraumatic stress disorder (PTSD) who have not found relief from traditional treatments such as therapy and medication.

Evidence reported demonstrates that SGB with an anesthetic has limited side effects, and is relatively safe when administered by a trained clinician. After the procedure, some patients who have been treated with SGB for PTSD symptoms find more benefit from traditional treatments like “talk” therapy because of a reduction in anxiety and other symptoms. SGB is not a cure, and the treatment does not work for everyone. However, it is a promising option for PTSD symptoms, for example, those which have not responded to other evidence-based treatments. Still, the duration of effect in anesthetic-mediated SGB is limited.

Disclosed embodiments comprise use of a neurotoxin to establish an SGB. Disclosed embodiments comprise administering a therapeutically effective amount of at least one neurotoxin into the SG or the vicinity thereof. In embodiments comprising injection into the SG, suitable compositions can comprise Clostridial neurotoxins, for example botulinum neurotoxins. Disclosed embodiments comprise combination treatments wherein a local anesthetic is applied to the SG. In embodiments, the neurotoxin can be administered along with an anesthetic. In embodiments, the neurotoxin can be administered along with an SSRI. In embodiments, the neurotoxin can be administered along with an SNRI.

### Definitions

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Fast-acting neurotoxin” as used herein refers to a botulinum toxin that produces effects in the patient more rapidly than those produced by, for example, a botulinum neurotoxin type A. For example, the effects of a fast-acting botulinum toxin (such as botulinum type E) can be produced within 36 hours.

“Fast-recovery neurotoxin” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as in the treatment of hyperhidrosis. However, the usual duration of an intramuscular injection of a botulinum neurotoxin type A is typically about 3 to 4 months.

“Neurotoxin” means a biologically active molecule with a specific affinity for a neuronal cell surface receptor. Neurotoxin includes Clostridial toxins both as pure toxin and as complexed with one to more non-toxin, toxin-associated proteins.

“Patient” means a human or non-human subject receiving medical or veterinary care.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Therapeutic formulation” means a formulation that can be used to treat and thereby alleviate a disorder or a disease and/or symptom associated thereof.

“Therapeutically effective amount” means the level, amount or concentration of an agent (e.g. such as a clostridial toxin or pharmaceutical composition comprising clostridial toxin) needed to treat a symptom, disease, disorder, or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of an symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®).

Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing a lyophilized powder for reconstituting in a suitable vehicle such as saline for injection.

In embodiments, the botulinum toxin is formulated in a solution containing saline and pasteurized Human Serum Albumin (HSA), which stabilizes the toxin and minimizes loss through non-specific adsorption. The solution can be sterile filtered (for example using a 0.2 μm filter), filled into individual vials, and then vacuum-dried to give a sterile lyophilized powder. In use, the powder can be reconstituted by, for example, the addition of sterile unpreserved normal saline (sodium chloride 0.9% for injection).

In an embodiment, botulinum type A is supplied in a sterile solution for injection with a 5-mL vial nominal concentration of 20 ng/mL in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects of the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABA_{A} receptor. The GABA_{A} receptor inhibits neuronal activity by effectively shunting current flow across the cell membrane. GABA_{A} receptor modulators may enhance the inhibitory effects of the GABA_{A} receptor and reduce electrical or chemical signal transmission from the neurons. Examples of GABA_{A} receptor modulators include benzodiazepines, such as diazepam, oxaxepam, lorazepam, prazepam, alprazolam, halazeapam, chordiazepoxide, and chlorazepate. Compositions can also contain glutamate receptor modulators that decrease the excitatory effects mediated by glutamate receptors. Examples of glutamate receptor modulators include agents that inhibit current flux through AMPA, NMDA, and/or kainate types of glutamate receptors. Further disclosed compositions comprise esketamine.

Disclosed neurotoxin compositions can be injected into the patient, for example using a needle or a needleless device. In certain embodiments, the method comprises sub-dermally injecting the composition in the individual. For example, administering may comprise injecting the composition through a needle of no greater than about 30 gauge. In certain embodiments, the method comprises administering a composition comprising a botulinum toxin type A.

Administration of the disclosed compositions can be carried out by syringes, catheters, needles and other means for injecting. The injection can be performed on any area of the mammal's body that is in need of treatment, however disclosed embodiments contemplate injection into the patient's head, specifically the SG. The injection can be into any specific area such as epidermis, dermis, fat, muscle, nerve junction, or subcutaneous layer.

More than one injection and/or sites of injection may be necessary to achieve the desired result. Also, some injections, depending on the location to be injected, may require the use of fine, hollow, Teflon®-coated needles. In certain embodiments, guided injection is employed, for example by electromyography, or ultrasound, or fluoroscopic guidance or the like.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the symptom or condition being treated.

Methods of Use

Methods disclosed herein can comprise administration of a neurotoxin, for example a Clostridial toxin, for example a botulinum type A, to a patient to prevent or alleviate the symptoms associated with a neurologic and/or psychiatric disorder. For example, disclosed methods can prevent or alleviate the occurrence of pain, nausea, vomiting, light sensitivity, sound sensitivity, acute stress, flashbacks, nightmares, severe anxiety, uncontrollable thoughts, irritability, angry outbursts, reckless or self-destructive behavior, being easily startled, lack of concentration, insomnia, and combinations thereof.

Disorders suitable for treatment with disclosed methods comprise, for example, Post-Traumatic Stress Disorder (PTSD), Depression, Anxiety, Headache, Post-Herpetic Neuralgia (PHN), Intractable Angina, arrhythmias, and Chronic Regional Pain Syndrome (CRPS), depressive disorder, major depressive disorders, bipolar disorder, acute stress disorder, generalized anxiety disorder, obsessive-compulsive disorder, social anxiety disorders, panic disorders, phobias, and trichotillomania.

Common PTSD co-morbidities which can be treated with disclosed methods include major depression, anxiety disorders, impulsivity/violent behavior, and substance abuse.

Disclosed embodiments can comprise the administration of a local anesthetic to the SG in combination with the neurotoxin administration. For example, 5 to 10 mL of a local anesthetic such as lidocaine 1 or 2% can be administered via injection to the SG, or to the vicinity of the SG.

Disclosed combination treatments (for example, an SSRI in combination with a neurotoxin in the case of PTSD treatment, or zoledronic acid in combination with a neurotoxin in the case of CRPS treatment) can provide a synergistic effect. In some embodiments, a monthly dose of zoledronic acid, is about 5000 mg or less, about 4000 mg or less, about 3000 mg or less, about 2000 mg or less, about 1000 mg or less, about 700 mg or less, about 600 mg or less, about 1 mg to about 4,000 mg, about 1 mg to about 1,000 mg, about 10 mg to about 1000 mg, about 50 mg to about 1000 mg, about 10 mg to about 600 mg, about 40 mg to about 600 mg, about 50 mg to about 600 mg, about 40 mg to about 400 mg, about 50 mg to about 200 mg, about 200 mg to about 300 mg, about 250 mg to about 350 mg, or about 100 mg to about 600 mg, about 40 mg to about 2000 mg, about 40 mg to about 800 mg, about 50 mg to about 800 mg, or about 100 mg to about 800 mg, about 40 mg to about 1000 mg, about 50 mg to about 1000 mg, or about 100 mg to about 1000 mg, or any monthly dose in a range bounded by, or between, any of these values.

SSRIs suitable for use in disclosed embodiments comprise, for example, Fluoxetine (Prozac), Sertraline (Zoloft), Paroxetine (Paxil), Escitalopram (Lexapro), Fluvoxamine (Luvox), Citalopram (Celexa), Volazodone (Viibrid), Vortioxetine (Brintellix), and combinations thereof. In embodiments, the dose of the SSRI can be, for example, 5 mg/day, 10 mg/day, 15 mg/day, 20 mg/day, 25 mg/day, 30 mg/day, or the like. In embodiments, the dose of the SSRI can be, for example, 5 mg every other day, 10 mg every other day, 15 mg every other day, 20 mg every other day, 25 mg every other day, 30 mg every other day, or the like.

SNRIs suitable use in disclosed embodiments comprise, for example, atomoxetine (Strattera), desvenlafaxine (Pristiq, Khedezla), duloxetine (Cymbalta, Irenka), levomilnacipran (Fetzima), milnacipran (Savella), tramadol (Ultram), venlafaxine (Effexor XR), In embodiments, the dose of the SNRI can be, for example, 5 mg/day, 10 mg/day, 15 mg/day, 20 mg/day, 25 mg/day, 30 mg/day, or the like. In embodiments, the dose of the SSRI can be, for example, 5 mg every other day, 10 mg every other day, 15 mg every other day, 20 mg every other day, 25 mg every other day, 30 mg every other day, or the like.

Disclosed treatment methods comprise use of a neurotoxin in combination with a local anesthetic, with both applied to the SG or the vicinity thereof.

Disclosed methods can comprise use of multiple clostridial neurotoxins.

Disclosed embodiments comprise combination treatments wherein a patient undergoes further therapy following neurotoxin administration to the SG. For example, following disclosed methods, a patient can be treated with Cognitive Processing Therapy, Eye Movement Desensitization and Reprocessing (EMDR), Cognitive Behavioral Therapy (CBT), yoga, acupuncture, and combinations thereof. Disclosed embodiments comprise establishing an SGB and prescribing an exercise regimen for the patient. Disclosed embodiments comprise establishing an SGB and prescribing a healthier diet for the patient. Disclosed embodiments comprise establishing an SGB and prescribing a yoga regimen for the patient.

Further embodiments comprise administering to a patient in need thereof a therapeutically effective amount of an agent that reduces the level or activity of ghrelin or ghrelin receptor, compared to before the agent is administered. In embodiments such agents can comprise a growth hormone secretagogue receptor 1a (GHSr1a) antagonist, a GHSr1a inverse agonist, or an agent that inhibits the activity of ghrelin O-acyltransferase (GOAT).

Disclosed embodiments comprise establishing an SGB and reducing the number or amount of other medications, for example anti-depression medication, prescribed to the patient.

Neurotoxin Dosages

The neurotoxin can be administered in an amount of between about 10⁻³ U/kg and about 35 U/kg. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 5 Units to about 200 Units of a neurotoxin, such as a botulinum type A, can be used and in another embodiment, from about 10 Units to about 100 Units of a neurotoxin, such as a botulinum type A, can be locally administered into a target tissue.

In embodiments, administration can comprise a total dose per treatment session of about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, administration can comprise a total dose per treatment session of not less than 10 Units of a neurotoxin, for example botulinum type A neurotoxin, or not less than 20 Units, or not less than 30 Units, or not less than 40 Units, or not less than 50 Units, or not less than 60 Units, or not less than 70 Units, or not less than 80 Units, or not less than 90 Units, or not less than 100 Units, or not less than 110 Units, or not less than 120 Units, or not less than 130 Units, or not less than 140 Units, or not less than 150 Units, or not less than 160 Units, or not less than 170 Units, or not less than 180 Units, or not less than 190 Units, or not less than 200 Units, or not less than 210 Units, or not less than 220 Units, or not less than 230 Units, or not less than 240 Units, or not less than 250 Units, or not less than 260 Units, or not less than 270 Units, or not less than 280 Units, or not less than 290 Units, or not less than 300 Units, or the like.

In embodiments, administration can comprise a total dose per treatment session of not more than 10 Units of a neurotoxin, for example botulinum type A neurotoxin, or not more than 20 Units, or not more than 30 Units, or not more than 40 Units, or not more than 50 Units, or not more than 60 Units, or not more than 70 Units, or not more than 80 Units, or not more than 90 Units, or not more than 100 Units, or not more than 110 Units, or not more than 120 Units, or not more than 130 Units, or not more than 140 Units, or not more than 150 Units, or not more than 160 Units, or not more than 170 Units, or not more than 180 Units, or not more than 190 Units, or not more than 200 Units, or not more than 210 Units, or not more than 220 Units, or not more than 230 Units, or not more than 240 Units, or not more than 250 Units, or not more than 260 Units, or not more than 270 Units, or not more than 280 Units, or not more than 290 Units, or not more than 300 Units, or the like.

In embodiments, administration can comprise a total dose per year of not more than 400 Units of a neurotoxin, for example botulinum type A neurotoxin, or not more than 500 Units, or not more than 600 Units, or not more than 700 Units, or not more than 800 Units, or not more than 900 Units, or not more than 1000 Units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms associated with the neurologic and/or psychiatric disorder. However, preferred embodiments comprise repeating the treatment prior to the return of symptoms. Therefore, disclosed embodiments comprise repeating the treatment, for example, after 6 weeks, 8 weeks, 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A kit for practicing disclosed embodiments is also encompassed by the present disclosure. The kit can comprise a 30 gauge or smaller needle and a corresponding syringe. The kit can also comprise a Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of PTSD

A PTSD patient is treated via injection of 35 U of botulinum type A into the stellate ganglion (SG) to establish a stellate ganglion block (SGB). The patient lies on their back with a pillow placed under their shoulder blades. The patient's neck is cleansed with an antiseptic soap. The doctor presses on the neck to identify where to place the needle. The patient is asked to avoid talking, coughing, or swallowing, as these activities may cause the needle to move.

The patient reports better sleep due to a reduction in stressful dreams. This reduction lasts for 12 weeks.

### Example 2

### Treatment of PTSD

A PTSD patient is treated via injection of 45 U of botulinum type A into the SG to establish an SGB.

The patient reports a reduction in stress-related headaches. This reduction lasts for 16 weeks.

### Example 3

### Treatment of PTSD

A PTSD patient is treated via injection of 60 U of botulinum type B into the stellate ganglion (SG) to establish a stellate ganglion block (SGB). The patient is also administered an SSRI.

The patient reports a reduction in their depression symptoms. This reduction lasts for 10 weeks.

### Example 4

### Treatment of PTSD

A PTSD patient is treated via injection of 40 U of botulinum type E as well as 2 mL of 1% lidocaine into the stellate ganglion (SG) to establish a stellate ganglion block (SGB). The patient is also administered an SNRI.

The patient reports a reduction in their depression symptoms. This treatment is repeated after 4 weeks.

### Example 5

### Treatment of Anxiety

A patient with anxiety is treated via injection of 40 U of botulinum type A into the stellate ganglion (SG) to establish a stellate ganglion block (SGB).

The patient reports a reduction in their anxiety symptoms. This reduction lasts for 10 weeks.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1) A method of treating a neurological or psychological disorder comprising the steps of administering a botulinum toxin into the stellate ganglion nerve of a mammal to treat at least one of PTSD, Depression, Anxiety, Headache and CRPS; thereby reducing the occurrence of at least one symptom of the disorder.

2) The method of claim 1, wherein the botulinum toxin is native immunotype A.

3) The method of claim 2, wherein the botulinum toxin is administered by injection.

4) A method of treating a neurological or psychological disorder comprising administering a botulinum toxin into the stellate ganglion in a subject with a neurological or psychological disorder to thereby treat their symptoms.

5) A method of treating a neurological or psychological disorder comprising administering a botulinum toxin into the stellate ganglion wherein the administration is performed without any imaging, by palpating the anterior transverse process of C6, or the Chassagne tubercle, and injecting the botulinum toxin medially.

6) A method of treating a neurological or psychological disorder comprising administering a botulinum toxin into the stellate ganglion wherein the administration is performed by imaging the treatment area, and injecting the botulinum toxin medially.

7) The method of claim 4, 5, or 6 wherein the botulinum toxin is a botulinum toxin type A.

8) The method of claim 4, 5, or 6 wherein the botulinum toxin is a botulinum toxin type B, C, E, or F.

9) The method of claim 7 or 8, wherein the total dose of the botulinum toxin is 50-250 Units.

10) The method of claim 7 or 8, wherein the total dose of the botulinum toxin is 25-150 Units.

11) The method of claim 9, wherein said botulinum toxin is type A.

12) The method of claim 9, wherein said botulinum toxin is type B.

13) The method of claim 9, wherein said botulinum toxin is type C.

14) The method of claim 9, wherein said botulinum toxin is type E.

15) The method of claim 9, wherein said botulinum toxin is type F.

16) The method of claim 1, wherein said neurological or psychological disorder comprises PTSD.

17) The method of claim 1, wherein said neurological or psychological disorder comprises Depression.

18) The method of claim 1, wherein said neurological or psychological disorder comprises Anxiety.

19) The method of claim 1, wherein said neurological or psychological disorder comprises CRPS.

20) The method of claim 1, wherein said neurological or psychological disorder comprises Headache.

21) A method of treating a neurological or psychological disorder comprising the steps of administering a botulinum toxin into the stellate ganglion nerve of a mammal to treat at least one of PTSD, Depression, Anxiety, Headache and CRPS; then administering a local anesthetic to the stellate ganglion nerve of said mammal, thereby reducing the occurrence of at least one symptom of the disorder.

22) The method of claim 21, wherein said local anesthetic comprises lidocaine.

23) The method of claim 21, wherein said botulinum toxin is type A.

24) The method of claim 21, wherein said botulinum toxin is type B.

25) The method of claim 21, wherein said botulinum toxin is type C.

26) The method of claim 21, wherein said botulinum toxin is type E.

27) The method of claim 21, wherein said botulinum toxin is type F.

28) The method of claim 21, wherein said neurological or psychological disorder comprises PTSD.

29) The method of claim 21, wherein said neurological or psychological disorder comprises Depression.

30) The method of claim 21, wherein said neurological or psychological disorder comprises Anxiety.

31) The method of claim 21, wherein said neurological or psychological disorder comprises CRPS.

32) The method of claim 21, wherein said neurological or psychological disorder comprises Headache.

33) A method for comparing the efficacy and safety of two different Botulinum toxins, comprising:
measuring a reduction of a neurological or psychological disorder symptom of an individual resulting from administration of a first botulinum neurotoxin;
measuring a reduction of a neurological or psychological disorder symptom of an individual resulting from administration of a second botulinum neurotoxin; and
comparing the reduction in symptoms to determine a difference between the first botulinum neurotoxin and the second botulinum neurotoxin.
